(19) United States
(12) Patent Application Publication
(10) Pub. No.: US 2017/0292798 A1
(43) Pub. Date: Oct. 12, 2017

(54) LEAK DETECTION IN HEAT EXCHANGER SYSTEMS

(71) Applicant: Fluor Technologies Corporation, Sugar Land, TX (US)

(72) Inventor: Vuk Jancic, Hoofddorp (NL)

(21) Appl. No.: 15/092,010

(22) Filed: Apr. 6, 2016

(51) Int. Cl. F28F 27/00; F28F 11/00; G08B 21/18; G01N 33/00; G01M 3/04; F28D 7/16; G01F 23/00

(52) U.S. Cl. CPC F28F 27/00; F28D 7/16; F28D 7/1607; F28F 11/00; G01F 23/0007; G01N 33/0009; G01M 3/04; G08B 21/182; F28F 2265/16

(57) ABSTRACT

A heat exchanger comprises an outer shell extending between axially opposed ends and having a first fluid inlet and a first fluid outlet, one or more tubes passing through the tubular shell, a collection vessel disposed in an upper surface of the outer shell or the first fluid outlet, and a level sensor configured to detect the presence of the second heat exchange fluid within the collection vessel. The first fluid inlet and the first fluid outlet provide a first fluid pathway for a first heat exchange fluid through the outer shell, and the one or more tubes are configured to provide a second fluid pathway for a second heat exchange fluid between a second fluid inlet and a second fluid outlet.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Various industries utilize heat exchangers to indirectly contact and heat and/or cool streams, thereby allowing energy to be recovered or transferred between streams. This has a number of advantages including the utilization of waste heat or the ability to remove heat from a desired stream as needed. As one example, an air cooling system may pass air through a heat exchanger to cool the air. The air can be cooled through indirect contact with various heat exchange fluids including water or another aqueous fluid. Within the heat exchanger, the two fluids being heat exchanged can be contacted through the wall of a divider such as a heat exchange tube, channel, or plate, which may keep the two heat exchange fluids separated within the heat exchanger itself.

### SUMMARY

In an embodiment, a heat exchanger comprises an outer shell extending between axially opposed ends and having a first fluid inlet and a first fluid outlet, one or more tubes passing through the tubular shell, a collection vessel disposed in an upper surface of the outer shell or the first fluid outlet, and a level sensor configured to detect the presence of the second heat exchange fluid within the collection boot. The first fluid inlet and the first fluid outlet provide a first fluid pathway for a first heat exchange fluid through the outer shell, and the one or more tubes are configured to provide a second fluid pathway for a second heat exchange fluid between a second fluid inlet and a second fluid outlet.

In an embodiment, a heat exchange method comprises indirectly contacting a first heat exchange fluid with a second heat exchange fluid within a heat exchanger, collecting at least one of the first heat exchange fluid or the second heat exchange fluid in a collection vessel, detecting the presence of the at least one of the first heat exchange fluid or the second heat exchange fluid with a level sensor, and generating an alarm indicative of a leak of the at least one of the first heat exchange fluid or the second heat exchange fluid within the heat exchanger. The first heat exchange fluid is at least partially immiscible with the second heat exchange fluid, and the first heat exchange fluid is denser than the second heat exchange fluid.

In an embodiment, a heat exchange system comprises a heat exchanger configured to indirectly contact a first heat exchange fluid with a second heat exchange fluid, a collection vessel in fluid communication with the heat exchanger, a level sensor within the collection vessel, wherein the level sensor is configured to detect a level of an interface between the first heat exchange fluid and the second heat exchange fluid, and a controller in signal communication with the level sensor. The first heat exchange fluid is at least partially immiscible with the second heat exchange fluid, and the collection vessel is in fluid communication with one of an upper portion of the heat exchanger or a lower portion of the heat exchanger. The controller is configured to generate an alarm when the level of the interface between the first heat exchange fluid and the second heat exchange fluid passes a threshold.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIGS. 1A and 1B are schematic illustrations of embodiments of heat exchangers having leak detection units associated therewith.

FIG. 2 is another schematic illustration of an embodiment of a heat exchanger having a leak detection unit associated therewith,

FIG. 3 is still another schematic illustration of an embodiment of a heat exchanger having a leak detection unit associated therewith.

FIG. 4 schematically illustrates an embodiment of a controller according to an embodiment.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The following brief definition of terms shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example;

The terms “about” or “approximately” or the like, when used with a number, may mean that specific number, or alternatively, a range in proximity to the specific number, as understood by persons of skill in the art field; and

If the specification states a component or feature “may,” “can,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

Within a heat exchanger, the two heat exchange fluids can be separated by the wall of a divider. If a leak develops that allows direct contact between the two heat exchange fluids, it can be difficult to detect. In general, the fluid at the higher pressure will tend to leak into the heat exchange fluid at the lower pressure. When the two fluids are at least partially immiscible, the detection of the leak at various downstream equipment can be difficult to detect and have a detection delay that can lead to issues with the overall process.

Described herein is a system and methods for detecting a leak within a heat exchanger that is exchanging heat between two heat exchange fluids that are at least partially immiscible. The term fluid can refer to any material that flows in response to a differential pressure including at least liquids and gases. hi addition to being at least partially immiscible, the two heat exchange fluids may generally have a difference in density such that the fluid leaking into the other heat exchange fluid can separate from the fluid due to the density differences. For example, when one fluid is a gas and the other heat exchange fluid is a liquid, the gas will tend to rise within the liquid when the two mix.

The leak detection unit described herein generally comprises a collection vessel having a level sensor to detect the interface between the two heat exchange fluids. For example, if the gas leaks into the liquid, a collection vessel can be disposed on an upper surface of the liquid outlet or slightly downstream of the liquid outlet to allow any gas in the fluid to be captured within the collection vessel. A level sensor within the collection vessel can be used to detect a drop in the liquid level to thereby indicate the presence of the gas in the collection vessel, which provides a quick and efficient indication of a potential leak within the heat exchanger.

In addition to the level sensor, an outlet valve can be used with the collection vessel to allow the captured gas to be released to reset the liquid level. In order to verify that a leak has occurred, a sample connection or gas analyzer can be used to test the gas collected within the collection vessel. If the composition of the gas matches that of the gas used as the heat exchange fluid, then a determination of the presence of a leak in the heat exchanger can be made. At that point, the heat exchanger can be serviced to locate and fix the leak, or otherwise replace the various components or the entire heat exchanger.

While described in some embodiments as being used with a first heat exchange fluid comprising a gas and a second heat exchange fluid comprising a liquid, the system can be used for any two heat exchange fluids that are at least partially immiscible and have a density difference. If a heaver fluid (e.g., a dense liquid) is used with a lighter fluid (e.g., a less dense liquid), the collection vessel can be placed on a lower surface of an outlet line. The level sensor can then be configured to detect a liquid level of the denser liquid, and the presence of the denser liquid in the less dense liquid collection vessel may indicate a leak in the heat exchanger. This may provide a relatively fast leak indication to allow the heat. exchanger to be shut down prior to allowing for extensive mixing of the two heat exchange fluids.

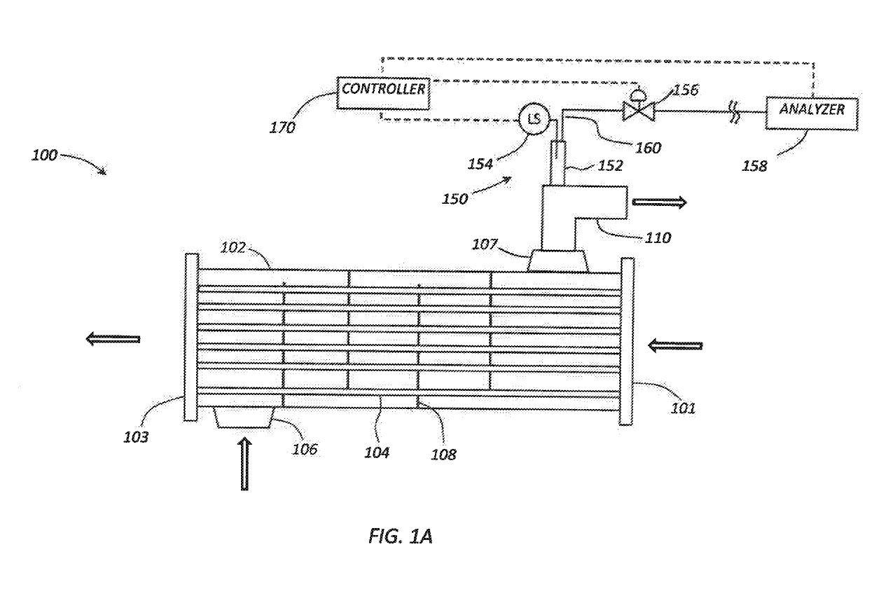

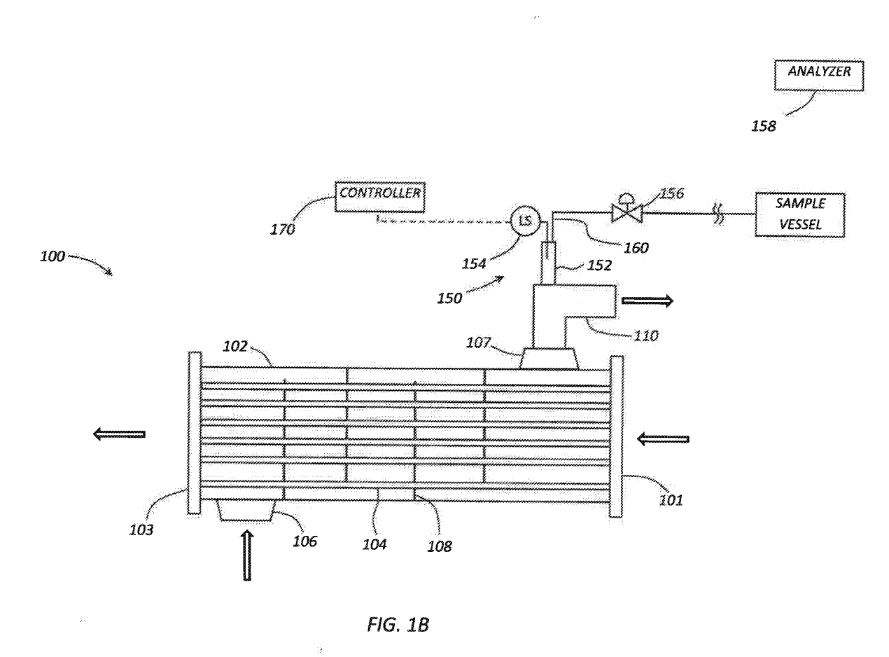

FIG. 1A schematically illustrates a heat exchanger system 100 having a leak detection unit 150 associated therewith, as described in more detail herein. The heat exchanger generally comprises an outer shell 102 having a plurality of end assemblies 101, 103. The end assemblies 101, 103 allow for a fluid tight connection to upstream and downstream piping carrying a first heat exchange fluid. A plurality of tubes or channels 104 can sealingly engage the end assemblies 101, 103 and provide a fluid pathway through the outer shell 102 for the first heat exchange fluid to pass through the heat exchanger 100. An inlet port 106 provides a fluid inlet for a second heat exchange fluid to pass into the outer shell 102 and contact an exterior of the tubes 104, thereby allowing for heat exchange between the first heat exchange fluid within the tubes 104 and the second heat exchange fluid outside of the tubes 104. A fluid outlet 107 provides an outlet for the second heat exchange fluid after the two heat exchange fluids are indirectly contacted within the heat exchanger 100.

The outer shell 102 can assume a number of shapes such as a cylindrical vessel. The outer shell 102 can be designed to retain the second heat exchange fluid at a desired pressure. In some embodiments, an optional layer of insulation can be disposed about the outer shell 102. The end assemblies provide for a sealed connection with the outer shell 102 to prevent the second heat exchange fluid from escaping through the ends of the heat exchanger 100 while also providing for a sealed connection with tubing 104 carrying the first heat exchange fluid. The end assemblies 101, 103 can take the form of flanges, threaded connections, welded connections, or the like. In some embodiments, only a single end assembly 101 may be present, and the other end assembly 103 may represent a head or cap. in this embodiment, the tubes 104 may enter and leave out of a single end assembly 101 or otherwise pass an inlet or outlet in the outer shell 102.

The tubes 104 can generally provide a fluid pathway between a first heat exchange fluid inlet and outlet so that a pathway is created for the first heat exchange fluid to pass through the heat exchanger 100. Since the heat exchange can occur through the wall of the tubes 104, the tubes 104 can be constructed of a material suitable for retaining the first heat exchange fluid at a working pressure while also providing a high heat transfer rate. Various metals such as steel, copper, aluminum, brass, and/or various corrosion resistant alloys can be used to form the tubes 104. While illustrated as having a single pass through the heat exchanger 100, the tubes 104 can assume a number of bends, turns, or loops within the heat exchanger 100 to increase their effective length within the outer shell 102.

One or more optional internal structures such as baffles 108 can be used to create an internal flow path for the second heat exchange fluid through the outer shell 102 between the inlet port 106 and the fluid outlet 107. The baffles 108 can create a series of sub-chambers within the outer shell 102 that direct the flow of the second heat exchange fluid along a flow path that has a longer length than the axial length of the outer shell 102 itself. This helps to create an increased contact time to provide additional heat exchange between the two heat exchange fluids, When present, the baffles 108 can also serve to support the tubes 104 within the outer shell 102.

In use, the first heat exchange fluid, such as a gas being cooled, can be passed through a first heat exchange fluid inlet (e.g., the first end assembly 101) and through the tubes 104 to a first heat exchange fluid outlet (e.g., the second end assembly 103). At the same time, a second heat exchange fluid, such as a liquid at a lower temperature than the first heat exchange fluid, can pass through the inlet port 106 and enter the outer shell 102. Within the outer shell 102, the baffles 108 can define a flow path for the second heat exchange fluid over the tubes 104. The overall fluid flowpath is depicted in FIG. 1A as being counter-current here the first heat exchange fluid generally flows in an opposite direction to the second heat exchange fluid. As the second heat exchange fluid flows over the tubes 104, the first heat exchange fluid can be cooled while the heat from the first heat exchange fluid is transferred to the second heat exchange fluid. Thus, the first heat exchange fluid passing out of the second end assembly 103 can be at a lower temperature than the first heat exchange fluid entering the heat exchanger at the first end assembly 101. Since the heat from the first heat exchange fluid is transferred to the second heat exchange fluid, the second heat exchange fluid entering the inlet port 106 can be at a lower temperature than the second heat exchange fluid passing out of the fluid outlet 107.

While depicted in FIG. 1A as a shell and tube heat exchanger with various features, any suitable heat exchanger can be used with the leak detection unit 150. For example, multi-pass shell and tube heat exchangers, plate or channel type heat exchangers, or the like, each in counter-current flow, co-current flow, cross-current flow, or any combination of flow types can also be used with the leak detection unit 150. Further, the heat exchanger 100 illustrated in FIG. 1A is shown in a horizontal configuration. However, vertical configurations or heat exchangers oriented at any other angles can be used with the leak detection unit 150.

The leak detection unit 150 generally comprises a collection vessel 152 disposed at or near an outlet conduit 110 of one of the heat exchange fluids in a position that is configured to collect the other heat exchange fluid if it is present. A level sensor 154 can be positioned to detect the first heat exchange fluid and/or the second heat exchange fluid within the collection vessel 152 to provide an indication of a leak within the heat exchanger 100. A controller 170 can be coupled to the level sensor 154 to process the level signal and generate various actions such as an alarm. As used in the discussion herein, the leaking fluid generally comprises the heat exchange fluid that is at a higher pressure and passes through a leak in the barrier between the two heat exchange fluids (e.g., the wall of the tubes 104, etc.) into the other heat exchange fluid within the heat exchanger 100. An outlet line 160 can be used with the collection vessel 152 to allow the collection vessel 152 to be reset if a leak is detected. A valve 156 can be used with the outlet line 160 to control the reset of the leak detection unit 150. An analyzer 158 can be used to detect a composition of the fluid in the collection vessel 152 to determine the composition of the fluid and/or confirm that the leaking fluid is one of the heat exchange fluids.

As noted above, the leak detection unit 150 generally detects a leak by measuring an accumulation of one fluid in the collection vessel 152 such that the leaking fluid displaces the heat exchange fluid within the collection vessel 152. In order for the fluid displacement to occur, the two heat exchange fluids should be at least partially immiscible so that they remain separate when in direct contact. The two heat exchange fluids can also exhibit a sufficient density difference to allow the leaking fluid to accumulate in the collection vessel 152 while being detectable by the level sensor 154.

The collection vessel 152 can comprise an extension or boot on an outlet of the heat exchange fluid having the lower operating pressure, though in some embodiments, the collection vessel 152 can alternatively or additionally be present on an outlet of the heat exchange fluid having a higher operating pressure (e.g., in the event of a pressure upset or imbalance, etc.). In general, the collection vessel 152 can comprise a relatively small vessel or extension disposed on one side of the heat exchange fluid outlet, The collection vessel 152 may generally have a volume that is sufficient to contain the level sensor and collect a sufficient volume of the potentially leaking heat exchange fluid for detection. In an embodiment, the collection vessel may have a volume between about 0.1 gallons to about 5 gallons (e.g., about 0.4 liters to about 19 liters), The collection vessel 152 can have any suitable shape and be constructed of any suitable material.

The collection vessel 152 can be positioned to capture the leaking fluid. When the leaking fluid has a lower density than the other heat exchange fluid into which it is leaking, the collection vessel 152 can be positioned on a top surface along the fluid outlet 107, the outlet conduit 110, along an upper surface of the outer shell 102, and/or along an upper surface of an outlet of the first exchange fluid. When the leaking fluid has a higher density than the other heat exchange fluid into which it is leaking, the collection vessel 152 can be positioned on a lower surface of the outlet conduit 110, along a lower surface of the outer shell 102, and/or along a lower surface of an outlet of the first heat exchange fluid.

The level sensor 154 is generally configured to detect the presence of the first heat exchange fluid, the second heat exchange fluid, or both within the collection vessel 152. In general, level sensors 154 tend to detect a fluid surface or interface with another fluid. As discussed herein, the level sensor 154 can be selected to detect an interface between the leaking fluid and the other heat exchange fluid surface.

In general, the level sensor 154 can comprise a continuous level sensor or a point level sensor. The continuous level sensor may provide an indication of the presence of the leaking fluid as well as the rate of collection of the leaking fluid in the collection vessel 152. This indication may provide a measurement of the leakage rate and/or the relative size of the leak, which may provide an indication of when an alarm should be triggered. A point-level sensor may provide an indication of when a threshold amount of the leaking fluid has accumulated within the collection vessel 152. Such a measurement may be suitable in some circumstances when detecting the presence of the leaking fluid is desired. Various types of continuous and/or point level sensors 154 can be used such as float sensors, pneumatic sensors, conductance sensors, visual level sensors, capacitance sensors, vibrating point sensors, or the like with the leak detection unit 150.

An outlet line 160 Can be fluidly coupled to the collection vessel 152 at or near the top of the collection vessel 152 when the leaking fluid is less dense than the other heat exchange fluid, or at or near the bottom of the collection vessel 152 when the leaking fluid is denser than the other heat exchange fluid. The leak detection system can be reset when a leak is detected by opening the outlet line 160 and allowing the leaked fluid to pass through the outlet line 160. The other heat exchange fluid can then refill the collection vessel 152 until a suitable level of the other heat exchange fluid is present in the collection vessel 152, which can be indicated by the level sensor 154. A valve 156, which can be manually or automatically controlled (e.g., by the controller 170), can be used to control the flow through the outlet line 160.

In some embodiments, the presence of a different heat exchange fluid in the collection vessel 152 may have a number of causes. For example, the presence of a gas in the collection vessel 152 illustrated in FIG. 1A could be due to the leakage of a gas from the tubes 104 or the entrainment of a gas in the second heat exchange fluid. In order to verify that the fluid present in the collection vessel 152 is a fluid that is leaking, a sample of the fluid present in h collection vessel 152 can be passed to an analyzer 158. The sample can be collected in a collection tank (e.g., a standalone tank or container), or the analyzer can be in fluid communication with the outlet line 160 and be configured to receive and process the fluid sample. Depending on the type of fluid expected to be present in the collection vessel 152, the analyzer 158 could comprise a liquid analyzer, a gas analyzer, or the like. Various suitable analyzers can be configured to detect one or more components of the fluid, and in some embodiments, may only be configured to detect one of the main components of the gas. Suitable analyzers can include, but are not limited to, a photoionization detector (PID), a thermal conductivity detector (TCD), a hydrogen flame detector, an ion mobility detector, a mass spectrometry detector, a spectral detector, or the like. A chromatographic or other separation device can be used within any of the analyzers to separate the flow of the components entering the analyzer 158.

A controller 170 can be in signal communication with the level sensor 154, the valve 156, and/or the analyzer 158. The controller 170 can generally be configured to receive the various signals such as the level sensor signal and provide processing and output of the signal for further use. The controller 170 can be coupled to various output devices such as alarms, control screens, or the like to provide an indication of the potential for the leak. When the controller 170 is in signal communication with the analyzer 158, the controller 170 can compare the output of the analyzer 158 with an expected composition of the leaking fluid to automatically determine if the fluid is leaking within the heat exchanger 100.

In some embodiments as shown in FIG. 1B, the valve 156 can be a manually operated valve. Further the valve 156 can allow the fluid to pass to a downstream collection point such as the analyzer 158 or a sample vessel or collection tank, which can be separately coupled to the analyzer 158. The ability to have a manual system may provide a low-cost option for detecting leaks and determining the source of the potential leaks.

Returning to FIG. 1A, the heat exchanger 100 and the leak detection unit 150 can be used to detect a leak of one heat exchange fluid into the other heat exchange fluid. In an embodiment, the first heat exchange fluid can comprise a fluid (e.g., a gas such as air, etc.) having a lower density than the second heat exchange fluid (e.g., a liquid such as water, etc.), and the first heat exchange fluid can be at a pressure that is greater than the pressure of the second heat exchange fluid. If a leak develops in one of the tubes 104 and/or a connection between one of the tubes and an end assembly 101, 103, the gas may tend to leak into the second heat exchange fluid.

When a leak occurs, the first heat exchange fluid can pass into the second heat exchange fluid within the outer shell 102. Since the two fluids are at least partially immiscible, a two-phase flow will develop. The first heat exchange fluid can then be carried with the second heat exchange fluid through the fluid outlet 107. The first heat exchange fluid can flow along an upper surface of the outlet conduit 110 and be captured in the collection vessel 152. As the first heat exchange fluid collects within the collection vessel 152, the first heat exchange fluid can displace the second heat exchange fluid within the collection vessel 152. The level sensor 154 can then detect when the level of the second heat exchange fluid drops to provide a signal to the controller 170. The signal can comprise a continuous level signal or a discrete level indication upon the level of the interface between the first heat exchange fluid and the second heat exchange fluid reaching a certain threshold. Upon detecting the signal, the controller 170 can provide an alarm signal to indicate the potential presence of a leak in the heat exchanger 100.

Once the leak is detected, a sample of the first heat exchange fluid in the collection vessel 152 can be passed through the outlet line 160, as controlled by valve 156, to the analyzer 158. The analyzer 158 can test the fluid sample to determine if the fluid has properties matching those of the first heat exchange fluid. If the fluid properties match, various actions can be taken such as shutting down the heat exchanger and performing maintenance to repair and/or replace the heat exchanger 100 or components thereof.

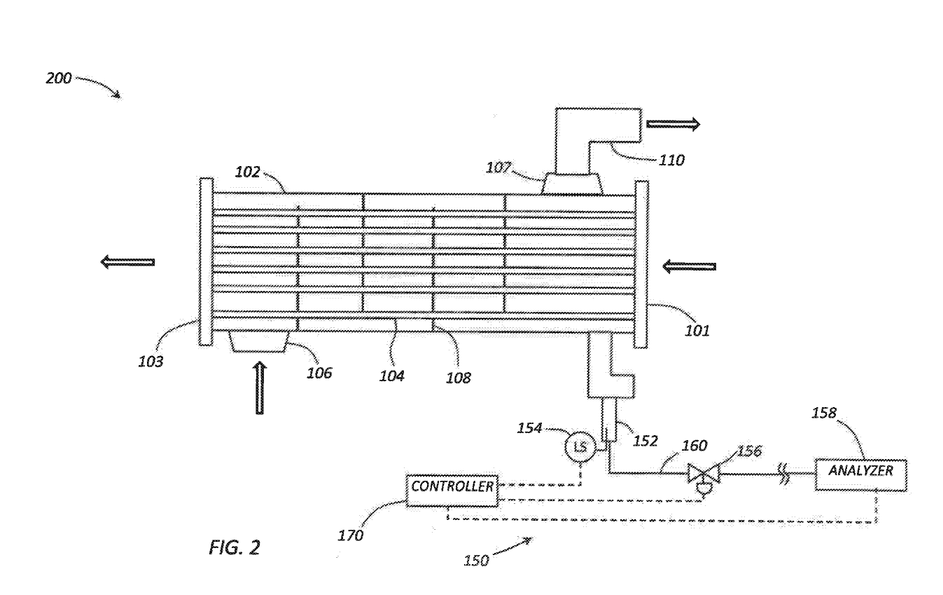

FIG. 2 illustrates a similar heat exchanger 200, and the similar elements will not be re-described in the interest of brevity. The main difference between the heat exchanger 100 described with respect to FIG. 1A and the heat exchanger 200, is the presence of the leak detection unit 150 being located on the lower surface of the outer shell 102 in order to detect the presence of a leaking fluid that has a higher density than the density of the second heat exchange fluid. As shown in FIG. 2, the collection vessel 152 can be located on a lower surface of the outer shell 102. In this embodiment, when the first heat exchange fluid comprises a denser fluid than the second heat exchange fluid, a leak of the first heat exchange fluid into the outershell 102 can result in the first heat exchange fluid accumulating in a lower portion of the outer shell 102. The first heat exchange fluid can then collect in the collection vessel 152 and be detected by the level sensor 154.

The use of the leak detection unit 150 in the heat exchanger 200 would operate in a similar manner as with the heat exchange system 100 illustrated in FIG. 1A. In an embodiment, as the first heat exchange fluid collects within the collection vessel. 152, the first heat exchange fluid can displace the second heat exchange fluid within the collection vessel 152. The level sensor 154 can then detect when the level of the first heat exchange fluid rises within the collection vessel 152 to provide a signal to the controller 170. Upon detecting the signal, the controller 170 can provide an alarm signal to indicate the potential presence of a leak in the heat exchanger 200.

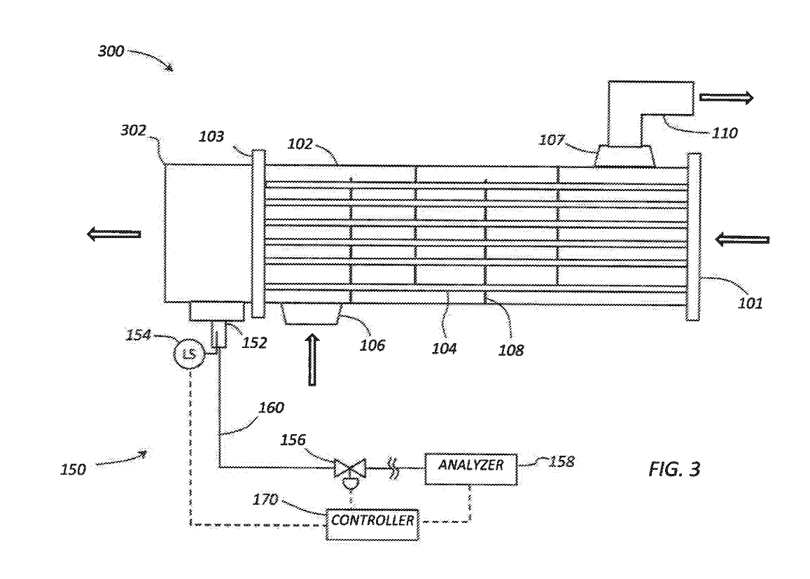

FIG. 3 illustrates still another heat exchanger 300 to the heat exchanger 200 described with respect to FIG. 2 and the heat exchanger 100 described with respect to FIG. 1A, and the similar elements will not be re-described in the interest of brevity. The main difference between the heat exchanger 300 and the heat exchangers 100, 200, is the presence of the leak detection unit 150 being located on the lower surface of an outlet conduit 302 associated with the first heat transfer fluid. in this embodiment, the leak detection unit 150 may be used to detect a leak of the second heat transfer fluid into the tubes 104. The collection vessel 152 can be located on a lower surface of the outlet conduit 302 when the second heat transfer fluid is denser than the first heat transfer fluid, or on an upper surface of the outlet conduit 302 when the second heat transfer fluid is less dense than the first heat transfer fluid.

In this embodiment, a leak of the second heat exchange fluid into the tubes 104 can result in the second heat exchange fluid accumulating in a lower portion of the outlet conduit 302. The second heat exchange fluid can then collect in the collection vessel 152 and be detected by the level sensor 154. Upon detecting the signal, the controller 170 can provide an alarm signal to indicate the potential presence of a leak in the heat exchanger 300.

While described in FIGS. 1-3 as having a single leak detection unit 150, more than one leak detection unit 150 can be disposed in any of the embodiments described herein. For example, a heat exchanger can have both an upper and lower leak detection unit present on the shell side and/or on an outlet conduit of the tube side of the heat exchanger. When a plurality of leak detection units is present, each leak detection unit can have a separate controller and a single controller that is part of a control system. Similarly, a common analyzer can be used, or one or more different analyzers may be present to test the different potential fluids.

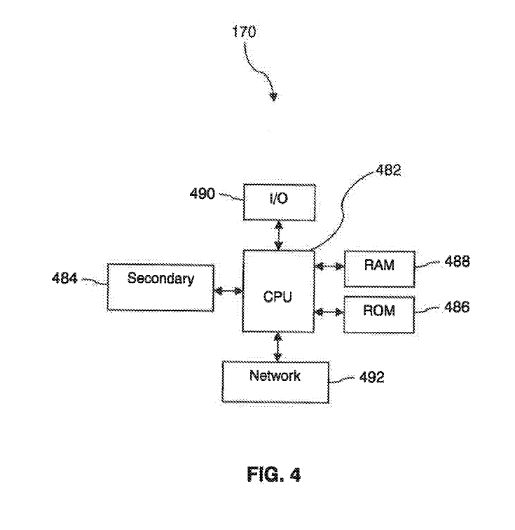

FIG. 4 illustrates an embodiment of the controller 170 suitable for implementing one or more embodiments disclosed herein. The controller 170 includes a processor 482 (which may be referred to as a central processor unit or CPU) that is in communication with memory devices including secondary storage 484, read only memory (ROM) 486, random access memory (RAM) 488, input/output (I/O) devices 490, and network connectivity devices 492. The processor 482 may be implemented as one or more CPU chips.

It is understood that by programming and/or loading executable instructions onto the controller 170, at least one of the CPU 482, the RAM 488, and the ROM 486 are changed, transforming the controller 170 in part into a particular machine or apparatus having the novel functionality taught by the present disclosure. It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

The secondary storage 484 is typically comprised of one or more disk drives or tape drives and is used for non-volatile storage of data and as an over-flow data storage device if RAM 488 is not large enough to hold all working data. Secondary storage 484 may be used to store programs which are loaded into RAM 488 when such programs are selected for execution. The ROM 486 is used to store instructions and perhaps data which are read during program execution. ROM 486 is a non-volatile memory device which typically has a small memory capacity relative to the larger memory capacity of secondary storage 484. The RAM 488 is used to store volatile data and perhaps to store instructions. Access to both ROM 486 and RAM 488 is typically faster than to secondary storage 484. The secondary storage 484, the RAM 488, and/or the ROM 486 may be referred to in some contexts as computer readable storage media and/or non-transitory computer readable media.

I/O devices 490 may include printers, video monitors, liquid crystal displays (LCDs), touch screen displays, keyboards, keypads, switches, dials, mice, track balls, voice recognizers, card readers, paper tape readers, or other well-known input devices.

The network connectivity devices 492 may take the form of moderns, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. These network connectivity devices 492 may enable the processor 482 to communicate with the Internet or one or more intranets. With such a network connection, it is contemplated that the processor 482 might receive information from the network, or might output information to the network in the course of performing the above-described method steps. Such information, which is often represented as a sequence of instructions to be executed using processor 482, may be received from and outputted to the network, for example, in the form of a computer data signal embodied in a carrier wave.

Such information, which may include data or instructions to be executed using processor 482 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several methods well known to one skilled in the art. The baseband signal and/or signal embedded in the carrier wave may be referred to in some contexts as a transitory signal.

The processor 482 executes instructions, codes, computer programs, scripts which it accesses from hard disk, floppy disk, optical disk (these various disk-based systems may all be considered secondary storage 484), ROM 486, RAM 488, or the network connectivity devices 492. While only one processor 482 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 484, for example, hard drives, floppy disks, optical disks, and/or other device, the ROM 486, and/or the RAM 488 may be referred to in some contexts as non-transitory instructions and/or non-transitory information.

In an embodiment, the controller 170 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the controller 170 to provide the functionality of a number of servers that is not directly bound to the number of computers in the controller 170. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third party provider. Some cloud computing environments may comprise cloud computing resources owned and operated by the enterprise as well as cloud computing sources hired and/or leased from a third party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, a magnetic disk, an optical disk, a solid state memory chip, for example analog magnetic tape, compact disk read only memory (CD-ROM) disks, floppy disks, jump drives, digital cards, multimedia cards, and others. The computer program product may be suitable for loading, by the controller 170, at least portions of the contents of the computer program product to the secondary storage 484, to the ROM 486, to the RAM 488, and/or to other non-volatile memory and volatile memory of the controller 170. The processor 482 may process the executable instructions and/or data structures in part by directly accessing the computer program product, for example by reading from a CD-ROM disk inserted into a disk drive peripheral of the controller 170. Alternatively, the processor 482 may process the executable instructions and/or data structures by remotely accessing the computer program product, for example by downloading the executable instructions and/or data structures from a remote server through the network connectivity devices 492. The computer program product may comprise instructions that promote the loading and/or copying of data, data structures, files, and/or executable instructions to the secondary storage 484, to the ROM 486, to the RAM 488, and/or to other non-volatile memory and volatile memory of the controller 170.

In some contexts, the secondary storage 484, the ROM 486, and the RAM 488 may be referred to as a non-transitory computer readable medium or a computer readable storage media. A dynamic RAM embodiment of the RAM 488, likewise, may be referred to as a non-transitory computer readable medium in that while the dynamic RAM receives electrical power and is operated in accordance with its design, for example during a period of time during which the controller 170 is turned on and operational, the dynamic RAM stores information that is written to it. Similarly, the processor 482 may comprise an internal RAM, an internal ROM, a cache memory, and/or other internal non-transitory storage blocks, sections, or components that may be referred to in some contexts as non-transitory computer readable media or computer readable storage media.

Having described numerous devices, systems, and method herein, various embodiments can include, but are not limited to:

In a first embodiment, a heat exchanger comprises an outer shell extending between axially opposed ends and having a first fluid inlet and a first fluid outlet, wherein the first fluid inlet and the first fluid outlet provide a first fluid pathway for a first heat exchange fluid through the outer shell; one or more tubes passing through the tubular shell, wherein the one or more tubes are configured to provide a second fluid pathway for a second heat exchange fluid between a second fluid inlet and a second fluid outlet; a collection vessel disposed in an upper surface of the outer shell or the first fluid outlet; and a level sensor configured to detect the presence of the second heat exchange fluid within the collection boot.

A second embodiment can include the heat exchanger of the first embodiment, wherein the first heat exchange fluid comprises a liquid, and wherein the second heat exchange fluid comprises a gas.

A third embodiment can include the heat exchanger of the second embodiment, wherein the first heat exchange fluid comprises cooling water, and wherein the second heat exchange fluid comprises air.

A fourth embodiment can include the heat exchanger of any of the first to third embodiments, wherein the second heat exchange fluid is less dense than the first heat exchange fluid.

A fifth embodiment can include the heat exchanger of any of the first to fourth embodiments further comprising: an outlet line in fluid communication with an upper portion of the collection vessel; and a valve configured to control flow through the outlet line.

A sixth embodiment can include the heat exchanger of any of the first to fifth embodiments, further comprising an analyzer, wherein the analyzer is configured to receive a sample of the second heat exchange fluid and provide an indication of the composition of the second heat exchange fluid.

A seventh embodiment can include the heat exchanger of any of the first to sixth embodiments, wherein the first fluid pathway is countercurrent to the second fluid pathway.

In an eighth embodiment, a heat exchange method comprises indirectly contacting a first heat exchange fluid with a second heat exchange fluid within a heat exchanger, wherein the first heat exchange fluid is at least partially immiscible with the second heat exchange fluid, and wherein the first heat exchange fluid is denser than the second heat exchange fluid; collecting at least one of the first heat exchange fluid or the second heat exchange fluid in a collection vessel; detecting the presence of the at least one of the first heat exchange fluid or the second heat exchange fluid with a level sensor; and generating an alarm indicative of a leak of the at least one of the first heat exchange fluid or the second heat exchange fluid within the heat exchanger.

A ninth embodiment can include the method of the eighth embodiment, wherein the at least one of the first heat exchange fluid or the second heat exchange fluid is the first heat exchange fluid, and wherein the collection vessel is located on a lower surface of the heat exchanger or a heat exchanger outlet.

A tenth embodiment can include the method of the eighth embodiment, wherein the at least one of the first heat exchange fluid or the second heat exchange fluid is the second heat exchange fluid, and wherein the collection vessel is located on an upper surface of the heat exchanger or a heat exchanger outlet.

An eleventh embodiment can include the method of any of the eighth to tenth embodiments, further comprising: selectively passing a portion of the at least one of the first heat exchange fluid or the second heat exchange fluid to an analyzer; and detecting a composition of the at least one of the first heat exchange fluid or the second heat exchange fluid with the analyzer.

A twelfth embodiment can include the method of the eleventh embodiment, wherein generating the alarm is based on the detecting of the composition of the at least one of the first heat exchange fluid or the second heat exchange fluid with the analyzer.

A thirteenth embodiment can include the method of any of the eighth to twelfth embodiments, further comprising: replacing or repairing the heat exchanger in response to the alarm.

A fourteenth embodiment can include the method of any of the eighth to thirteenth embodiments, wherein the heat exchanger comprises a shell and tube heat exchanger, a plate type heat exchanger, or a channel type heat exchanger.

In a fifteenth embodiment, a heat exchange system comprises a heat exchanger configured to indirectly contact a first heat exchange fluid with a second heat exchange fluid, wherein the first heat exchange fluid is at least partially immiscible with the second heat exchange fluid; a collection vessel in fluid communication with the heat exchanger, wherein the collection vessel is in fluid communication with one of an upper portion of the heat exchanger or a lower portion of the heat exchanger; a level sensor within the collection vessel, wherein the level sensor is configured to detect a level of an interface between the first heat exchange fluid and the second heat exchange fluid; and a controller in signal communication with the level sensor, wherein the controller is configured to generate an alarm when the level of the interface between the first heat exchange fluid and the second heat exchange fluid passes a threshold.

A sixteenth embodiment can include the heat exchange system of the fifteenth embodiment, wherein the heat exchange comprises a shell and tube heat exchanger, wherein the first heat exchange fluid is on a tube side of the heat exchanger, and wherein the second heat exchange fluid is on a shell side of the heat exchanger.

A seventeenth embodiment can include the heat exchange system of the sixteenth embodiment, wherein the first heat exchange fluid is less dense than the second heat exchange fluid, and wherein the collection vessel is disposed on the upper portion of the shell side of the heat exchanger.

An eighteenth embodiment can include the heat exchange system of the sixteenth embodiment, wherein the first heat exchange fluid is denser than the second heat exchange fluid, and wherein the collection vessel is disposed on the lower portion of the shell side of the heat exchanger.

A nineteenth embodiment can include the heat exchange system of the sixteenth embodiment, wherein the first heat exchange fluid is less dense than the second heat exchange fluid, and wherein the collection vessel is disposed on the lower portion of the tube side of the heat exchanger.

A twentieth embodiment can include the heat exchange system of the sixteenth embodiment, wherein the first heat exchange fluid is denser than the second heat exchange fluid, and wherein the collection vessel is disposed on the upper portion of the tube side of the heat exchanger.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above, but is defined by the claims which follow, that scope including all equivalents of the subject matter of the claims. Each and every claim is incorporated as further disclosure into the specification and the claims are embodiment(s) of the present invention(s). Furthermore, any advantages and features described above may relate to specific embodiments, but shall not limit the application of such issued claims to processes and structures accomplishing any or all of the above advantages or having any or all of the above features.

Additionally, the section headings used herein are provided for consistency with the suggestions under 37 C.F.R. 1.77 or to otherwise provide organizational cues. These headings shall not limit or characterize the inventions) set out in any claims that may issue from this disclosure. Specifically and by way of example, although the headings might refer to a “Field,” the claims should not be limited by the language chosen under this heading to describe the so-called field. Further, a description of a technology in the “Background” is not to be construed as an admission that certain technology is prior art to any invention(s) in this disclosure. Neither is the “Summary” to be considered as a limiting characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure, but should not be constrained by the headings set forth herein.

Use of broader terms such as “comprises,” “includes,” and “having” should be understood to provide support for narrower terms such as “consisting of,” “consisting essentially of,” and “comprised substantially of.” Use of the terms “optionally,” “my,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A heat exchanger comprising:
an outer shell extending between axially opposed ends and having a first fluid inlet and a first fluid outlet, wherein the first fluid inlet and the first fluid outlet provide a first fluid pathway for a first heat exchange fluid through the outer shell;
one or more tubes passing through the tubular shell, wherein the one or more tubes are configured to provide a second fluid pathway for a second heat exchange fluid between a second fluid inlet and a second fluid outlet;
a collection vessel disposed in an upper surface of the outer shell or the first fluid outlet; and
a level sensor configured to detect the presence of the second heat exchange fluid within the collection vessel.

2. The heat exchanger of claim 1, wherein the first heat exchange fluid comprises a liquid, acid wherein the second heat exchange fluid comprises a gas.

3. The heat exchanger of claim 2, wherein the first heat exchange fluid comprises cooling water, and wherein the second heat exchange fluid comprises air.

4. The heat exchanger of claim 1, wherein the second heat exchange fluid is less dense than the first heat exchange fluid.

5. The heat exchanger of claim 1, further comprising:
an outlet line in fluid communication with an upper portion of the collection vessel; and
a valve configured to control flow through the outlet line.

6. The heat exchanger of claim 1, further comprising an analyzer, wherein the analyzer is configured to receive a sample of the second heat exchange fluid and provide an indication of the composition of the second heat exchange fluid.

7. The heat exchanger of claim 1, wherein the first fluid pathway is countercurrent to the second fluid pathway.

8. A heat exchange method comprising:
indirectly contacting a first heat exchange fluid with a second heat exchange fluid within a heat exchanger, wherein the first heat exchange fluid is at least partially immiscible with the second heat exchange fluid, and wherein the first heat exchange fluid is denser than the second heat exchange fluid;
collecting at least one of the first heat exchange fluid or the second heat exchange fluid in a collection vessel;
detecting the presence of the at least one of the first heat exchange fluid or the second heat exchange fluid with a level sensor; and
generating an alarm indicative of a leak of the at least one of the first heat exchange fluid or the second heat exchange fluid within the heat exchanger.

9. The method of claim 8, wherein the at least one of the first heat exchange fluid or the second heat exchange fluid is the first heat exchange fluid, and wherein the collection vessel is located on a lower surface of the heat exchanger or a heat exchanger outlet.

10. The method of claim 8, wherein the at least one of the first heat exchange fluid or the second heat exchange fluid is the second heat exchange fluid, and wherein the collection vessel is located on an upper surface of the heat exchanger or a heat exchanger outlet.

11. The method of claim 8, further comprising:
selectively passing a portion of the at least one of the first heat exchange fluid or the second heat exchange fluid to an analyzer; and
detecting a composition of the at least one of the first heat exchange fluid or the second heat exchange fluid with the analyzer.

12. The method of claim 11, wherein generating the alarm is based on the detecting of the composition of the at least one of the first heat exchange fluid or the second heat exchange fluid with the analyzer.

13. The method of claim 8, further comprising:
replacing or repairing the heat exchanger in response to the alarm.

14. The method of claim 8, wherein the heat exchanger comprises a shell and tube heat exchanger, a plate type heat exchanger, or a channel type heat exchanger.

15. A heat exchange system comprising:
a heat exchanger configured to indirectly contact a first heat exchange fluid with a second heat exchange fluid, wherein the first heat exchange fluid is at least partially immiscible with the second heat exchange fluid;
a collection vessel in fluid communication with the heat exchanger, wherein the collection vessel is in fluid communication with one of an upper portion of the heat exchanger or a lower portion of the heat exchanger;
a level sensor within the collection vessel, wherein the level sensor is configured to detect a level of an interface between the first heat exchange fluid and the second heat exchange fluid; and
a controller in signal communication with the level sensor, wherein the controller is configured to generate an alarm when the level of the interface between the first heat exchange fluid and the second heat exchange fluid passes a threshold.

16. The system of claim 15, wherein the heat exchanger comprises a shell and tube heat exchanger, wherein the first heat exchange fluid is on a tube side of the heat exchanger, and wherein the second heat exchange fluid is on a shell side of the heat exchanger.

17. The system of claim 16, wherein the first heat exchange fluid is less dense than the second heat exchange fluid, and wherein the collection vessel is disposed on the upper portion of the shell side of the heat exchanger.

18. The system of claim 16, wherein the first heat exchange fluid is denser than the second heat exchange fluid, and wherein the collection vessel is disposed on the lower portion of the shell side of the heat exchanger.

19. The system of claim 16, wherein the first heat exchange fluid is less dense than the second heat exchange fluid, and wherein the collection vessel is disposed on the lower portion of the tube side of the heat exchanger.

20. The system of claim 16, wherein the first heat exchange fluid is denser than the second heat exchange fluid, and wherein the collection vessel is disposed on the upper portion of the tube side of the heat exchanger.
